(19) United States
(12) Patent Application Publication
(10) Pub. No.: US 2023/0148965 A1
(43) Pub. Date: May 18, 2023

(54) ADHESION DETECTION FOR A MEDICAL PATCH

(71) Applicant: Given Imaging LTD., Yoqneam (IL)

(72) Inventors: Eyal David, Atlit (IL); Iddo Diukman, Zihron Yaakov (IL); Baruch Gruman, Lapid (IL)

(21) Appl. No.: 17/917,021

(22) PCT Filed: May 20, 2021
(86) PCT No.: PCT/IL2021/050582

§ 371 Date: Oct. 5, 2022

Related U.S. Application Data
(60) Provisional application No. 63/028,838, filed on May 22, 2020.

(51) Int. Cl. A61B 5/00; A61B 5/07

(52) U.S. Cl. CPC A61B 5/6843; A61B 5/6833; A61B 5/073; A61B 1/041

(57) ABSTRACT

A patch configured for being applied to a patients skin, said patch comprising a contact area configured for being in direct contact with the patients skin when applied thereto, and a detector configured for detecting contact between the contact area and the patients skin.

### TECHNOLOGICAL FIELD

The present invention is in the field of medical patches, in particular, medical patches configured for communicating with in-vivo devices.

### BACKGROUND OF THE INVENTION

Medical patches (also referred as skin patches) are devices configured for being fitted to a patient's skin for one of two main purposes: medicative and monitoring. Medicative patches, commonly referred to as transdermal patches, comprise a medication and are configured for providing this medication to the patient via the skin, either by puncturing the skin (with a needle) or by transdermal diffusion. Monitoring patches, one the other hand, are configured for sensing different parameters of the patient (e.g. micro-movements, electricity, pulse etc.) and for communicating with in-vivo devices as very common with pacemakers.

Medical patches may be applied to the skin in various ways, one way being an adhesive layer.

Acknowledgement of the above references herein is not to be inferred as meaning that these are in any way relevant to the patentability of the presently disclosed subject matter.

### GENERAL DESCRIPTION

In accordance with one aspect of the subject matter of the present application, there is provided a patch configured for being applied to a patient's skin, said patch comprising a contact area configured for being in direct contact with the patient's skin when applied thereto, and a detector configured for detecting contact between the contact area and the patient's skin.

The patch may further comprise an indicator module associated with the detector and configured, based on input therefrom, to indicate states of contact of the contact area with the patient's skin. In particular, the detector may provide at least one of the following:
a positive contact indication signal configured indicating that the contact area is in adequate contact with the patient's skin; and
a negative contact indication signal configured for indicating that the contact area is not in adequate contact with the patient's skin.

In addition to the above, the indicator module may comprise several indication signals, each relating to a different state of contact between the contact area and the patient's skin. In accordance with a specific example, the contact area may comprise two or more different regions and the indicator may be configured for providing, for at least some of each of these regions a positive/negative indication signal.

The patch of the present application can thus alert the patient or a medical practitioner monitoring the patient regarding a malfunction. In case of the such malfunction, the patient or medical practitioner may either reattach the patch such that the contact area is properly fitted to the patient's skin or replace the patch.

The detector may be based on any one of the following mechanisms, but not limited thereto: electric current, electric capacitance, electric induction, heat capacitance and chemical reaction.

In accordance with a specific implementation, the patch may comprise a communication module configured for providing communication between the patch and an in-vivo device located within the patient. The communication module may be further configured for providing communication with one or more ex-vivo devices. The communication module may comprise a power source and an antenna arrangement configured for providing the above desired communication.

In accordance with a specific example, the in-vivo device may be a swallowable endoscopic capsule configured for providing data regarding the patient's GI. Under this example, the communication patch may be configured for staying in communication with a movable in-vivo device traversing the patient's GI. As such, detachment of the patch from the patient's skin may impede communication between the patch and the capsule, which may, under extreme circumstances, make the entire endoscopic procedure useless. In addition, noting that such a procedure (or indeed any endoscopic/colonoscopic procedure) requires a substantial preparation of the patient (laxatives, bowel cleaning etc.) which is usually unpleasant, providing a direct indication that something is wrong may spell the difference between proper completion of the procedure and going through the preparation a second time.

The patch may comprise a first layer with a rear face constituting the contact area, and a front face facing away from the patient. In accordance with one example, the first layer may have embedded therein the communication module and additional patch components. In accordance with another example, the patch may comprise additional layers configured for accommodating the communication module and any additional patch components.

Under a specific arrangement, the patch is designed such that none of the electrical components of the patch are in direct contact with the patient's skin. Under this arrangement, for example, the detector may be a capacitance detector. The capacitance detector is configured for measuring the electrical capacitance and providing said reading to the indicator module or to a processor which is, in turn, associated with the indicator module.

The capacitance detector may be calibrated to have a baseline reading corresponding to a state in which the contact area is fully detached from the patient's skin. Thus, when the patch is properly adhered to the patient's skin, the capacitance detector will detect a spike in capacitance compared to the baseline reading, and when a portion of the contact area becomes detached from the patient's skin, the capacitance detector will detect a drop in capacitance.

It has been discovered that detachment of even a portion of the contact area from the patient's skin can drastically affect the capacitance measured by the capacitance detector, which allows providing an accurate monitoring mechanism for proper attachment of the patch to the patient's skin.

It should be appreciated that the distance at which degradation of the sensitivity of the capacitance detector depends on various parameters, including, but not limited to the initial distance for which the baseline sensitivity is calibrated to, size of the sensor and the SNR. In accordance with a particular example of the present application, the sensor size and SNR may be calibrated such that any increase of the initial distance by more than at least 30% will yield a significant change in capacitance, allowing its detection. As a specific example, the initial distance of the sensor from the skin may range between 1.5-5 mm, more particularly between 2-4 mm, and even more particularly around 3 mm.

The baseline can be chosen, for example, as the baseline capacitance when the patch is fully adhered to the patient's skin. Alternatively, the baseline may also be chosen as the baseline capacitance when the patch is completely detached from the patient's skin (e.g. surrounded by air).

The capacitance detector may be connected to the antenna arrangement of the communication module and utilize components of the antenna arrangement as part of the detector. The capacitance detector may operate under the following scheme:

In general, the scheme may support more than one capacitive sensor. Each capacitive sensor consists of a sensing electrode connected to an oscillator, and a reference electrode connected to the ground plane of the circuit. The shape and the distance between electrodes may vary and depend on use case and sensitivity optimization.

Each oscillator, when enabled, generates a square wave signal at its output. The frequency of the square wave may vary in a certain range, inverse-proportional to the sensing capacitor value. The oscillator output signal, chosen by the selector, is used as a counter clock. Before each measurement the counter is reset and then enabled for a constant time window. At the end of the window the counter readout is proportional to the oscillator frequency and inverse-proportional to the sensor capacitance. The circuit is calibrated with 2 known capacitors, so the offset and the slope constants are recorded in NVM (Non Volatile Memory). Using these constants and the counter readout, the CPU calculates the real capacitance measured by the sensor.

Alternatively, the capacitance detection may also be performed by Self capacitance, which is relative to earth ground. In order to measure the self capacitance, charge is transferred between three difference capacitors. First, the charge stored on a Vreg capacitor (recommended value of 1 uF), is used to charge an external unknown capacitance during the charge phase. Second, the charge from the external capacitance is transferred to an internal sampling capacitor. During this transfer phase when charge is moved from the external capacitor to the sample capacitor the Vreg capacitor is refilled with charge by an LDO. These charge and transfer phases are repeated until the voltage on the internal sampling capacitor changes by the desired amount. This voltage can be changed to allow for a wide range of external capacitances.

The contact area may comprise an adhesive layer configured for allowing fitting of the patch to the patient's skin. In accordance with a specific example of the subject matter of the present application, the adhesive material may be chosen such that it provides, on the one hand, the required adhesion between the patch and the patient's skin, and, on the other hand, the required dielectric properties allowing the capacitance detector to properly distinguish between different adhesion states of the patch. Examples of the adhesive material which may be used may include, but are not limited to:

The capacitive sensor may reside on the inner side of the foam layer, closer to the patient's skin and separated therefrom by the adhesive layer alone, or, alternatively, on the outer side of the foam layer, distanced from the patient's skin, or any other position therebetween.

In accordance with another design example, the detection of adhesion to the patient's skin may be performed based on load resistance and a resonance capacitor. Specifically, the patch may comprise an antenna coil and a resonance capacitor at working frequency. Under this configuration, the load to the antenna driver is pure active resistance composed mainly by losses caused by human tissue attachment of the antenna coil.

Detaching the patch from the body decreases the human tissue loss, and therefore decreases the load resistance of the drive amplifier. This resistance change may be used to monitor the attachment of the patch to the body.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1 is a schematic front view of a patch in accordance with and embodiment of the present application, when applied to a patient's abdominal region;

FIG. 2A is a schematic front view of the patch shown in FIG. 1;

FIG. 2B is a schematic exploded view of the patch shown in FIG. 2A;

FIG. 3 is a schematic diagram of a capacitance detector implemented in the patch shown in FIGS. 1 to 2B;

FIG. 4; is a schematic graph showing readings taken by the capacitance detector when attached and detached from the body;

FIG. 5 is a schematic diagram of another example of a capacitance detector which can be used in the patch shown in FIGS. 1 to 2B; and

FIG. 6 is a schematic diagram of yet another example of a capacitance detector which can be used in the patch shown in FIGS. 1 to 2B.

It will be appreciated that for simplicity and clarity of illustration, elements shown in the figures have not necessarily been drawn accurately or to scale. For example, the dimensions of some of the elements may be exaggerated relative to other elements for clarity, or several physical components may be included in one functional block or element. Further, where considered appropriate, reference numerals may be repeated among the figures to indicate corresponding or analogous elements.

### DETAILED DESCRIPTION OF EMBODIMENTS

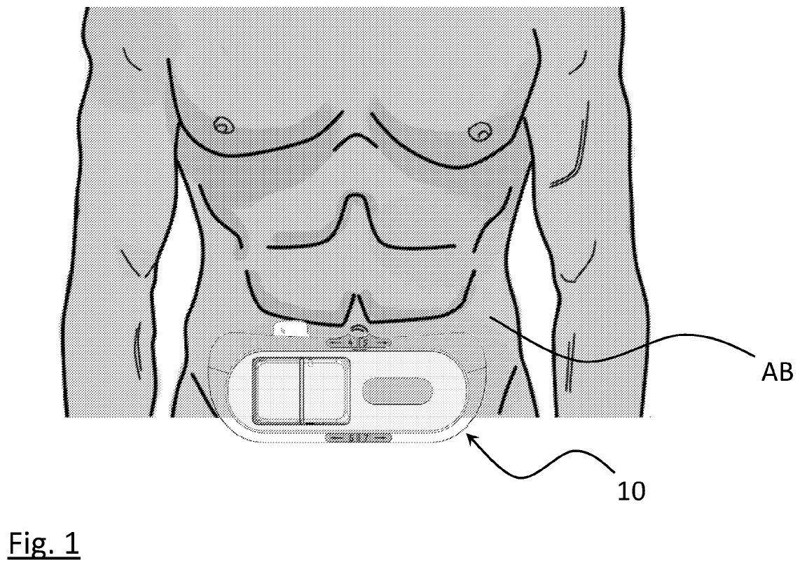

Attention is first drawn to FIG. 1 in which a patch, generally designated 10 is shown adhered to a patient's abdominal region AB, and constituting a part of a diagnostic system 1, further comprising an in-vivo device IV (not shown) configured for being introduced into the patient's gastrointestinal system. As can be seen, the patch is fitted just below the naval of the patient and covers a substantial portion of the bottom abdominal region thereof.

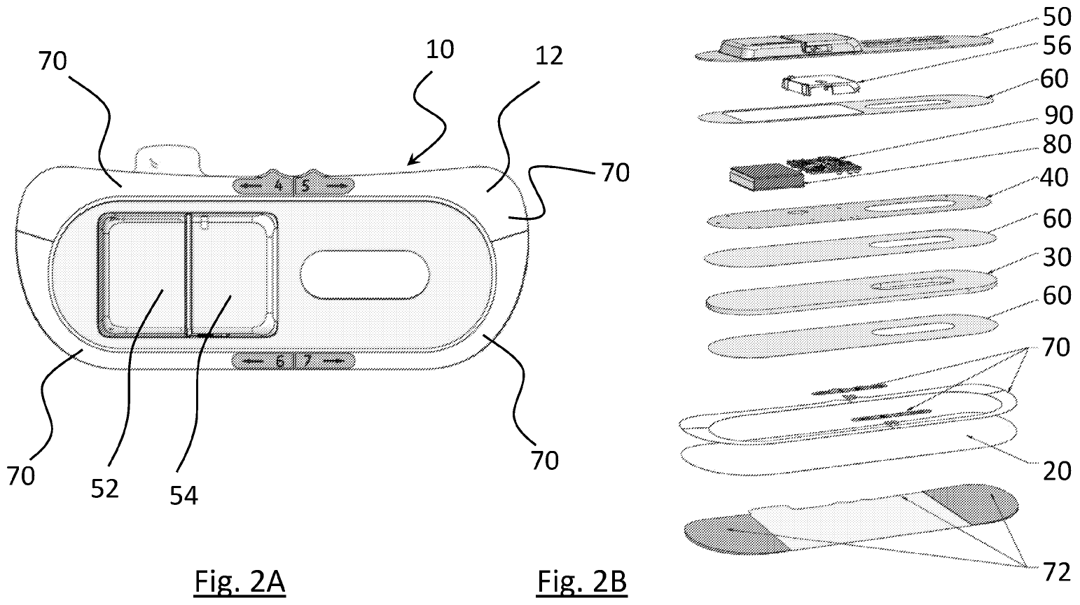

With additional reference being made to FIGS. 2A and 2B, the patch 10 comprises a patch body 12 consisting of a plurality of layers including (but not limited to):
an adhesive layer 20 configured for being in direct contact with the patient's body and for fixating the position of the patch with respect to the patient's body;
a spacer layer 30 configured for distancing any electrical components of the patch 10 from the patient's skin;
a communication layer 40 in the form of a printed antenna;
an external cover layer 50;
two intermediate adhesive layers 60; and
a plurality of removable films 70.

The patch 10 further comprises a power unit 80 and a processing unit 90 nested within respective inclusions 52 and 54 of the external cover layer 50.

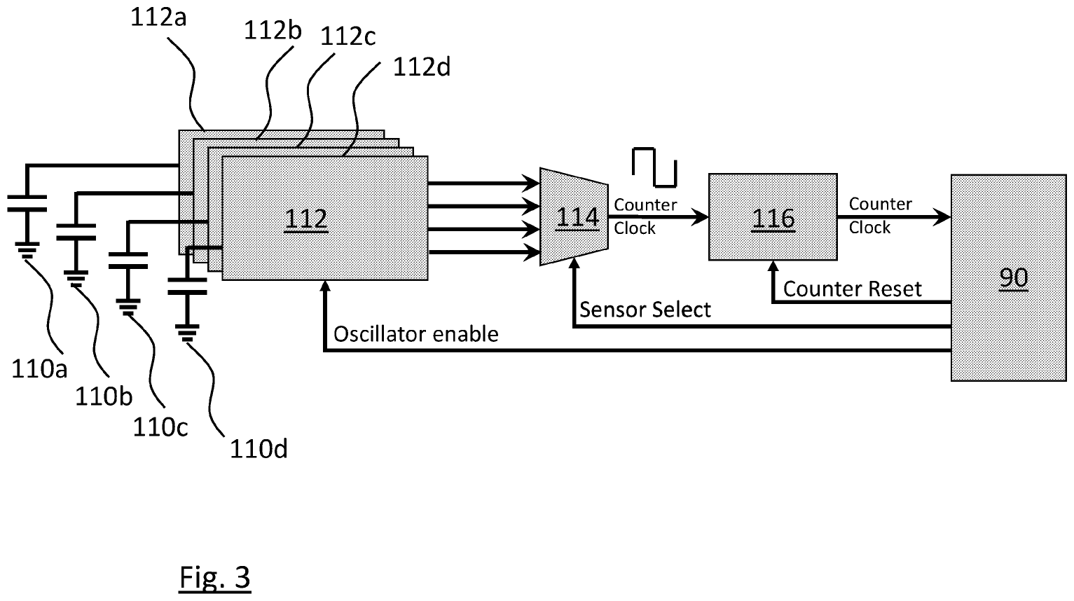

The communication layer 40 comprises a sensor arrangement (shown in FIG. 3) which, in conjunction with processing unit 90 form, inter alia, a capacitance detecting arrangement configured for monitoring the electrical capacitance between the patch 10 and the patient's skin. In particular, the capacitance detection arrangement of the present invention is sensitive to the distance between the patch 10 and the patient's skin, whereby monitoring capacitance allows alerting the patient and/or health care practitioner regarding full/partial detachment of the patch 10 from the skin. It should be noted that since the patch 10 is configured for being in constant communication with the in-vivo device IV, full/partial detachment of the patch 10 from the skin may greatly affect communication with the in-vivo device IV and the patch's 10 ability of receiving/sending signals to and from the in-vivo device IV respectively.

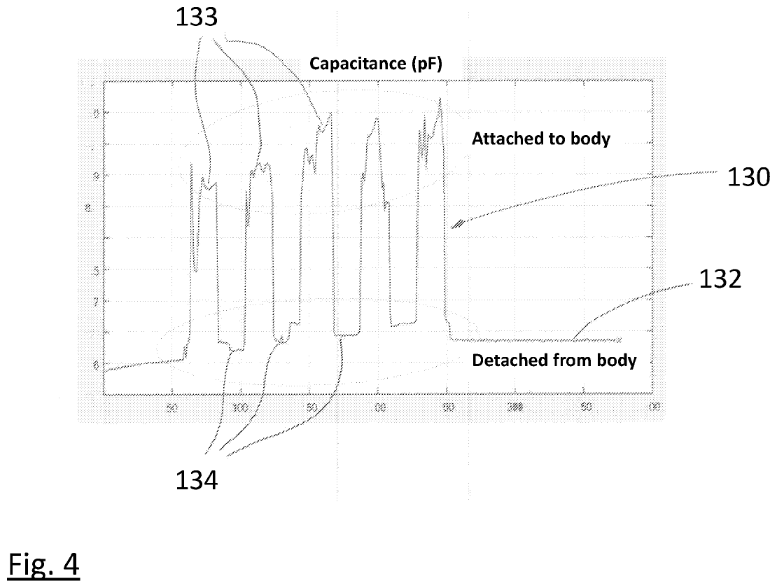

With additional reference being made to FIG. 4, a graph is shown, generally designated 130, demonstrating the capacitance measured by the sensor arrangement when the patch 10 is attached/detached from the patient's skin. The graph 130 is shown where the horizontal axis denotes time (in seconds), and the vertical axis denotes the capacitance (in pico-Farads).

When the patch 10 is completely detached from the patient's body, the sensor arrangement provides a baseline reading 132. The graph 130 represents experimental data yielded when the patch was alternately fitted and removed from the patient's skin. As can be seen, when the patch 10 is properly fitted to the patient's skin, the capacitance spikes up to peaks 133, ranging between 8.8 to 10.3 pF, while, when the patch 10 is detached from the patient's skin, capacitance drops to troughs 134, ranging between 6.2 to 6.7 pF.

This change in capacitance is sufficiently significant in order to detect during operation of the patch 10, whereby the patient or healthcare practitioner may be alerted to the fact via a variety of signals, including (but not limited to): light, vibration, text message, sound etc.

Reverting to FIG. 3, the implementation of the capacitance detection arrangement 100 is shown comprising four capacitance sensors 110a to 110d, each connected to respective capacitance sensing oscillators 112a to 112d. The capacitance sensors 110a to 110d may be positioned at different locations along the patch 10, thereby allowing individual monitoring of adhesion of said locations to the patient's skin. In particular, such an arrangement allows alerting the patient not only to the fact that the patch 10 is detached from the body, but also indicate which portion of the patch 10 became detached.

The capacitance sensing oscillators 112a to 112d are coupled to a selector 114, configured for selecting an output signal from the oscillators 112a to 112d in order to sample each of the capacitance sensors periodically and individually.

The arrangement is such that each oscillator 112, when enabled, generates a square wave signal 115 at its output. The frequency of the square wave 115 may vary in a certain range, inverse-proportional to the sensing capacitor 110 value. The oscillator output signal, chosen by the selector 114, is used as a counter. Before each measurement the counter is reset and then enabled for a constant time window. At the end of the window the counter readout is proportional to the oscillator frequency and inverse-proportional to the sensor capacitance. The circuit is calibrated with two known capacitors, so the offset and the slope constants are recorded in Non Volatile Memory (NVM). Using these constants and the counter readout, the CPU 90 calculates the real capacitance measured by the sensor 110, and can then perform the following:
If the capacitance monitored indicates lower values corresponding to the expected reading of the patch 10 when detached, the CPU 90 may send out a signal to activate the alert mechanism, indicating, to the user, that there is a problem;
If the capacitance monitored indicates high capacitance values corresponding to the expected reading of the patch 10 when properly placed, no action is taken.

In accordance with different variations of the present application, the capacitance sensors 110 of the sensor arrangement 100 can be placed inside the spacing layer 30, externally to the spacing layer (i.e. such that the spacing layer 30 is intermediate between the sensor arrangement 100 and the patient's skin, or even internally to the spacing layer 30.

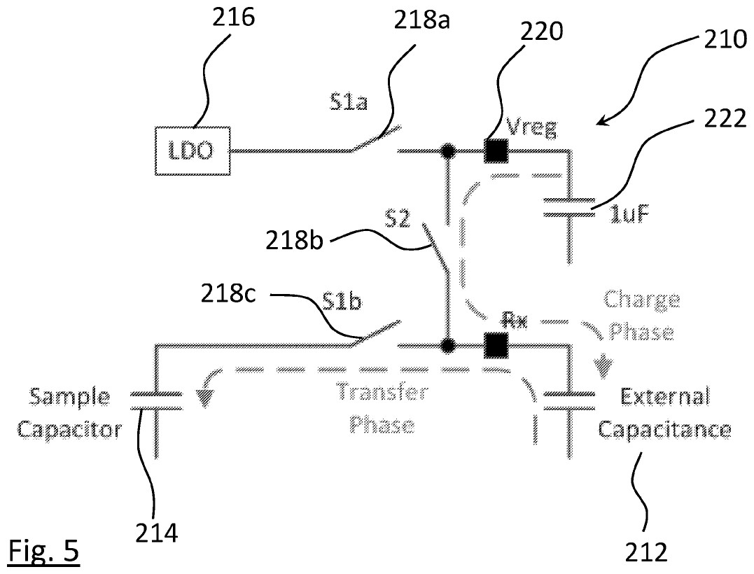

Further attention is drawn to FIG. 5, where another example of as capacitance arrangement is shown, generally designated 200, which is based on self capacitance, which is the capacitance relative to earth ground. In order to measure the self capacitance, charge is transferred between three difference capacitors—an external capacitor 212, an internal sampling capacitor 214 and a Vreg capacitor 222. First, the charge stored on the Vreg capacitor 222 (recommended value of 1 uF), is used to charge the external unknown capacitance 212 during the charge phase. Second, the charge from the external capacitance 212 is transferred to an internal sampling capacitor 214. During this transfer phase when charge is moved from the external capacitor 212 to the sample capacitor 214 the Vreg capacitor 222 is refilled with charge by the LDO 216. These charge and transfer phases are repeated until the voltage on the internal sampling capacitor 214 changes by the desired amount.

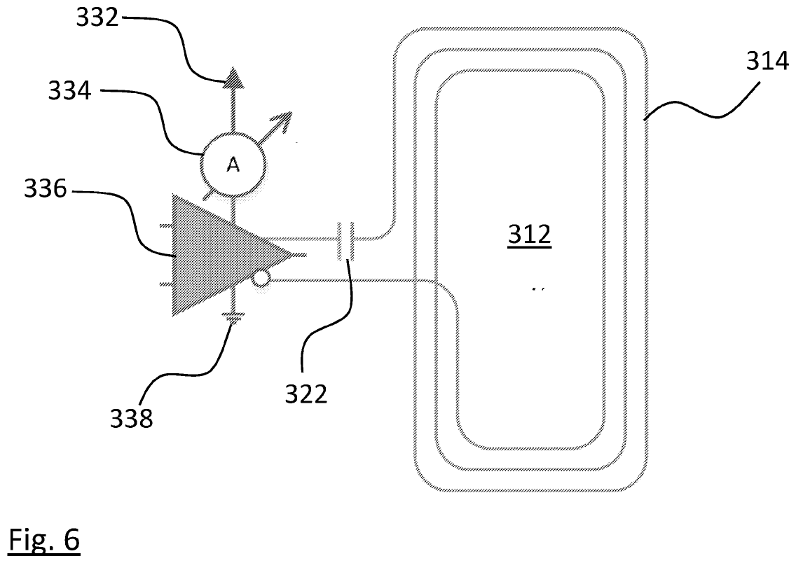

Attention is now drawn to FIG. 6, in which another example of an adhesion detection is shown, generally designated 300, being based on a communication antenna 310. Specifically, the patch comprises a downlink channel used for transmitting commands from the patch to the capsule. The Downlink antenna 310 is in the form of a coil 312 of several turns 314 located close to the perimeter of the patch flexible PCB, with a resonance capacitor 320 at the working frequency.

Due to the resonance capacitor 320, the load introduced to the antenna driver is pure active resistance, with no reactive component. This resistance is mostly composed by losses caused by human tissue attachment of the antenna coil 312. Detaching the patch from the body decreases the human tissue loss, and therefore decreases the load resistance of the drive amplifier. This resistance change may be used for detecting detachment of the patch from the body.

The load resistance measurement may be implemented by measuring the current consumption of the antenna driver. Assuming that the driver is a switching voltage source, the current consumption is inverse proportional to the load resistance. Current rise above a certain threshold may be used as a detachment indication.

Those skilled in the art to which this invention pertains will readily appreciate that numerous changes, variations, and modifications can be made without departing from the scope of the invention, mutatis mutandis.

1. A patch configured for being applied to skin of a person, the patch comprising:
a contact layer having a contact area configured for being in direct contact with the skin of the person when applied thereto;
a communication module configured for communications with an in-vivo device; and
a detector configured to detect contact between the contact area and the skin of the person,
wherein the contact layer separates all electrical components of the patch from the skin of the person such that none of the communication module and the detector are in direct contact with the skin of the person.

2. A patch according to claim 1, further comprising an indicator module associated with the detector and configured, based on input therefrom, to indicate states of contact of the contact area with the skin of the person.

3. (canceled)

4. A patch according to claim 2, wherein the indicator module comprises several indication signals, each relating to a different state of contact between the contact area and the skin of the person.

5. A patch according to claim 2, wherein the contact area comprises two or more different regions and the indicator is configured for providing, for at least some of each of these regions, a positive/negative indication signal.

6. A patch according to claim 1, wherein detection is based on any one of the following mechanisms: electric induction, heat capacitance, or chemical reaction.

7. (canceled)

8. A patch according to claim 1, wherein the communication module is further configured to communicate with one or more ex-vivo devices.

9. A patch according to claim 8, wherein the communication module comprises a power source and an antenna,
wherein detection of the contact between the contact area and the skin of the person is performed based on load resistance to a resonance capacitor electrically coupled to the antenna.

10. A patch according to claim 1, wherein the in-vivo device is a swallowable endoscopic capsule configured for providing data regarding a gastrointestinal tract of the person.

11. A patch according to claim 1, wherein the contact layer comprises a rear face constituting the contact area and a front face facing away from the person.

12-14. (canceled)

15. A patch according to claim 1, wherein the detector is a capacitance detector.

16. A patch according to claim 15, wherein the capacitance detector is configured for measuring electrical capacitance and providing a reading to the indicator module or to a processor which is associated with an indicator module.

17. A patch according to claim 16, wherein the capacitance detector is calibrated to have a baseline reading corresponding to a state in which the contact area is fully detached from the skin of the person.

18. A patch according to claim 17, wherein, when the contact area is properly adhered to the skin of the person, the capacitance detector will detect a spike in capacitance compared to the baseline reading, and when a portion of the contact area becomes detached from the skin of the person, the capacitance detector will detect a drop in capacitance.

19. A patch according to claim 16, wherein sensor size and signal-to-noise ratio are calibrated such that any increase of distance by more than at least 30% will yield a significant change in capacitance, allowing its detection.

20. A patch according to claim 16, wherein an initial distance of the capacitance detector from the skin ranges between 1.5-5 mm, more particularly between 2-4 mm, and even more particularly around 3 mm.

21. A patch according to claim 16, wherein the capacitance detector comprises a plurality of capacitive sensors.

22. A patch according to claim 21, wherein the capacitance detector further comprises:
a plurality of oscillators corresponding to the plurality of capacitive sensors, each oscillator of the plurality of oscillators connected to a respective capacitive sensor of the plurality of capacitive sensors and configured to output a respective oscillating signal;
a selector configured to select one of the respective oscillating signals to provide an oscillator output signal; and
a counter configured to reset prior to a measurement time window and to provide a counter readout based on the oscillator output signal for the measurement time window.

23. A patch according to claim 16, wherein capacitance detection is performed by self capacitance, which is relative to earth ground.

24. (canceled)

25. (canceled)

26. A patch according to claim 1, wherein the detector is the sole sensor in the patch configured to sense parameters of the person.

27. A patch consisting essentially of:
a plurality of layers comprising a contact layer having a contact area;
a detector, positioned in at least one layer of the plurality of layers, configured to detect contact between the contact area and skin of a person;
a communication module, positioned in at least one layer of the plurality of layers, configured to provide communication of data;
at least one processor, positioned in at least one layer of the plurality of layers, configured to process the data; and
a power source, positioned in at least one layer of the plurality of layers, configured to provide power to the detector, the communication module, and the at least one processor.
